(19) United States
(12) Patent Application Publication
(10) Pub. No.: US 2022/0332693 A1
(43) Pub. Date: Oct. 20, 2022

(54) SEPARATION OF CANNABINOIDS FROM MIXTURES THEREOF BY EXTRACTION AND DISTILLATION

(71) Applicant: CANOPY GROWTH CORPORATION, Smiths Falls (CA)

(72) Inventors: Christopher ADAIR, Smiths Falls (CA); Ben GEILING, Smiths Falls (CA); Logan MARYNISSEN, Smiths Falls (CA)

(21) Appl. No.: 17/631,583

(22) PCT Filed: Jul. 30, 2020
(86) PCT No.: PCT/CA2020/051050

§ 371 Date: Jan. 31, 2022

Related U.S. Application Data
(60) Provisional application No. 62/881,032, filed on Jul. 31, 2019.

(51) Int. Cl. C07D 311/80; C07C 37/76; B01D 1/22; B01D 3/14; B01D 11/04

(52) U.S. Cl. CPC C07D 311/80; C07C 37/76; B01D 1/225; B01D 3/148; B01D 11/0492

(57) ABSTRACT

The present disclosure relates to isolating one or more cannabinoids from an input mixture. There is disclosed an apparatus that comprises a mixing vessel, a volatizing unit, and a distillation unit. The mixing vessel combines a first input mixture and a high boiling-point carrier agent to generate a second input mixture. The volatizing unit volatilizes cannabinoids from the second input mixture for separating the mixture into a cannabinoid-containing vapor stream and a residue. The distillation unit receives the cannabinoid-containing vapor stream and separates a first cannabinoid from at least a second cannabinoid. There are also disclosed methods that comprise the steps of combining a first input mixture with a high boiling-point carrier agent to provide a second input mixture, volatilizing the second input mixture into a vapor stream containing one or more cannabinoids and a residue, and separating a first cannabinoid from a second in the distillation unit.

### CROSS-REFERENCE TO RELATED APPLICATION

This application claims priority to and benefit of U.S. Provisional Patent Application Ser. No. 62/881,032 filed on Jul. 31, 2019, which is hereby incorporated by reference.

### TECHNICAL FIELD

The present disclosure generally relates to the isolation of cannabinoids and mixtures of cannabinoids from an input mixture. In particular, the present disclosure relates to separation of cannabinoids from an input mixture to isolate pure compounds or purified mixtures.

### BACKGROUND

Naturally available cannabinoids, such as phytocannabinoids that are derived from plants, are typically sourced as a mixture of various cannabinoid compounds. Other plant components include, but are not limited to, lipids such as triglycerides and waxes. Due to the complexity of the mixture, it is challenging to fully understand the pharmacologic effect of individual cannabinoids and mixtures thereof. Furthermore, isolating individual cannabinoids from a source mixture has also proven challenging. Known methods for separating cannabinoids from each other in a mixture of cannabinoids include chromatography, short path distillation, and crystallization.

In chromatography, an input material is dissolved in a solvent to form a mobile phase. The mobile phase passes through a chromatography column. When the mobile phase containing the dissolved mixture passes through the chromatography column, the compounds within the mobile phase can travel at different speeds. The amount of time it takes for a compound to travel through the column is called retention time and it is a result of size, shape, total charge, hydrophobic groups present on the compound's surface, and binding capacity with the chromatography column. A widely used chromatography method for purification of molecules is High Pressure Liquid Chromatography (HPLC). This method involves passing the mobile phase through a chromatography column under pressures ranging from 10-400 atm and at a high flow rate. During HPLC, the use of small particles and the application of a high pressure increases the separation of the compounds within the mobile phase, which results in a shorter analysis time. However, HPLC requires complex and expensive equipment, generates significant waste, and is costly because of the large quantities of organic solvent required. Generally speaking, organic solvents present specific and further challenges because they can be expensive, toxic if consumed, and they are typically highly flammable and, therefore, they require specific equipment to handle safely. Other forms of chromatography, such as flash chromatography, simulated moving bed chromatography, and centrifugal partition chromatography can all suffer from similar challenges as HPLC with respect to complexity and solvent requirements.

Short path distillation involves a distillate travelling a short distance and it is typically performed under high vacuum to allow for the separation of larger heat-sensitive compounds. This method can run continuously and has the advantage of not using solvents to achieve the desired separation. However, short path distillation relies on a significant difference in vapor pressure between the compounds being separated. While this vapor pressure difference may hold true between cannabinoid compounds, as a whole, versus other components of plant-derived mixtures, the separation of individual cannabinoids from each other, or even from some waxes and oils, is not readily achievable by short path distillation.

Crystallization is another methodology that is known for separating cannabinoids from a mixture. Crystallization is possible with some cannabinoid compounds in the neutral form (primarily cannabigerol and cannabidiol) and the carboxylic acid form of other cannabinoids. Crystallization is challenging because it often takes considerable time. Crystallization also uses organic solvent and it is typically only able to produce isolates/single compounds—not complex mixtures. Furthermore, crystallization is traditionally a batch style process, which limits its utility or even prevents its integration into a continuous processing method. Crystallization also requires a high purity input prior to beginning the process in order to maximize yields. This means that an alternate separation technology is typically used before crystallization, which results in an overall inefficiency in the separation methodology.

Each of the known methodologies for separating cannabinoids from a mixture are challenged by inefficient and incomplete separation of desired compounds from the mixture, high operational costs and, often times, the necessity of using organic solvents. However, given the potential pharmacological value of individual cannabinoids, improved separation methods to provide highly pure individual cannabinoids—or mixtures of cannabinoids with specific relative ratios of one compound to another compound—may be desirable.

### SUMMARY

The present disclosure relates to apparatuses and methods for isolating one or more cannabinoids from an input mixture.

In an embodiment, the present disclosure relates to an apparatus for isolating one or more cannabinoids from an input mixture, the apparatus comprising: (a) a mixing vessel that is configured to receive a first input mixture and to combine the first input mixture with a high boiling-point carrier agent to provide a second input mixture, wherein one or more cannabinoids in the first input mixture are extracted into the high boiling-point carrier agent in the second input mixture; (b) a volatizing unit that is configured to receive and volatilize the second input mixture into a cannabinoid-containing vapor stream and a residue; and (c) a distillation unit for receiving the cannabinoid-containing vapor stream, wherein the distillation unit is configured to separate a first cannabinoid within the cannabinoid-containing vapor stream from at least a second cannabinoid.

In an embodiment, the present disclosure relates to an apparatus for isolating one or more cannabinoids from an input mixture, the apparatus comprising: (a) a volatizing unit that is configured to receive and volatilize a first input mixture into a cannabinoid-containing vapor stream and a residue; (b) a mixing vessel that is configured to receive and to combine the cannabinoid-containing vapor stream with a high boiling-point carrier agent to provide a second input mixture, wherein one or more cannabinoids are extracted into the high boiling-point carrier agent in the second input mixture; and (c) a distillation unit for receiving the second input mixture, wherein the distillation unit is configured to separate a first cannabinoid within the second input mixture from at least a second cannabinoid.

In an embodiment, the present disclosure relates to a method of isolating one or more cannabinoids from a input mixture, the method comprising steps of: (a) combining a first input mixture with a high boiling-point carrier agent to provide a second input mixture, wherein one or more cannabinoids from the first input mixture are extracted into the high boiling-point carrier agent in the second input mixture; (b) volatilizing the second input mixture to provide a cannabinoid-containing vapor stream and a residue; (c) conducting the cannabinoid-containing vapor stream to a distillation unit that separates a first cannabinoid within the cannabinoid- containing vapor stream from at least a second cannabinoid; and (d) collecting a product comprising the first cannabinoid.

In an embodiment, the present disclosure relates to a method of isolating one or more cannabinoids from an input mixture, the method comprising steps of: (a) introducing a first input mixture into a volatizing unit for separating the first input mixture into a cannabinoid-containing vapor stream and a residue; (b) combining the cannabinoid-containing vapor stream with a high boiling-point carrier agent to provide a second input mixture, wherein one or more cannabinoids from the cannabinoid-containing vapor stream are extracted into the high boiling-point carrier agent in the second input mixture; (c) conducting the second input mixture to a distillation unit that separates a first cannabinoid within the second input mixture from at least a second cannabinoid; and (d) collecting a product that comprises the first cannabinoid.

In some embodiments, the first input mixture is a cannabis resin.

In some embodiments, the volatizing unit is a wiped film evaporator.

In some embodiments, the distillation unit is a fractional distillation unit.

Without being bound by any particular theory, combining the input mixtures or cannabinoid-containing vapor streams with a high boiling-point carrier agent can generate a non-azeotropic mixture in which the boiling points of one or more of the cannabinoids in the mixture are altered. This change in boiling points may aid in the separation of individual cannabinoids from the mixture by distillation. Additionally, combining cannabinoid-containing concentrates with a high boiling-point carrier agent can reduce the viscosity of the concentrates, which can ease the handling of such resins or concentrates.

### BRIEF DESCRIPTION OF DRAWINGS

These and other features of the present disclosure will become more apparent in the following detailed description in which reference is made to the appended drawings. The appended drawings illustrate one or more embodiments of the present disclosure by way of example only and are not to be construed as limiting the scope of the present disclosure.

FIG. 1 shows a schematic representation of an apparatus, according to an embodiment of the present disclosure, which comprises a mixing vessel and a volatizing unit in fluid communication with a distillation unit.

FIG. 2 shows a schematic representation of an embodiment of the distillation unit shown in FIG. 1, wherein the unit includes devices to control temperature and pressure and a plenum that houses column packing materials.

FIG. 3 shows a schematic representation of another apparatus, according to an embodiment of the present disclosure that includes a preliminary treatment unit that is upstream of the volatizing unit.

FIG. 4 shows a schematic representation of a preliminary treatment, according to at least one embodiment of the present disclosure, for use with the apparatus shown in FIG. 3.

FIG. 5 shows two schematic representations of methods according to the present disclosure, wherein FIG. 5A shows the steps of a first method; and FIG. 5B shows the steps of a second method.

FIG. 6 shows a line graph that represents the residue product (RP) ratio of CBD:THC plotted over the various distillation temperatures for Example 5.

FIG. 7 shows a line graph that represents the net weight versus the temperature of the residue and the distillate for Example 6.

FIG. 8 shows data for Example 7, wherein FIG. 8A shows a line graph that represents the enrichment of CBD:THC over the four trials; and, FIG. 8B shows a line graph that represents the percentage of CBD (upper line) and THC (lower line) in the distillate product.

FIG. 9 shows a histogram that represents the distillate net output compared with the pressure within the distillation unit for Example 8.

FIG. 10 shows a histogram that represents the distillate/residue split as compared to the feed rate for Example 8.

FIG. 11 shows a line graph that represents the distillate/residue split as compared to the various temperatures tested for Example 9.

### DETAILED DESCRIPTION

Embodiments of the present disclosure relate to at least one apparatus and at least one method for separating one or more cannabinoids from an input mixture, such as a cannabis concentrate or a cannabis resin. Embodiments of the present disclosure relate to combining the input mixture with a high boiling-point carrier agent. The volatizing unit may be volatized and in a vapor state, or not. This combination may allow for some or all of the cannabinoids to be extracted from the volatizing unit into the high boiling-point carrier agent. The embodiments of the present disclosure may be suitable for use on an industrial scale and may have the advantage of operating continuously while substantially reducing or avoiding the use of organic solvents.

In one aspect, the present disclosure relates to an apparatus for isolating one or more cannabinoids from an input mixture.

In an embodiment, the present disclosure relates to an apparatus for isolating one more cannabinoids from an input mixture, the apparatus comprising: (a) a mixing vessel that is configured to receive a first input mixture and to combine the first input mixture with a high boiling-point carrier agent to provide a second input mixture, wherein one or more cannabinoids in the first input mixture are extracted into the high boiling-point carrier agent in the second input mixture; (b) a volatizing unit that is configured to receive and volatilize the second input mixture into a cannabinoid-containing vapor stream and a residue; and (c) a distillation unit for receiving the cannabinoid-containing vapor stream, wherein the distillation unit is configured to separate a first cannabinoid within the cannabinoid-containing vapor stream from at least a second cannabinoid.

In an embodiment, the present disclosure relates to an apparatus for isolating one or more cannabinoids from an input mixture, the apparatus comprising: (a) a volatizing unit that is configured to receive and volatilize a first input mixture into a cannabinoid-containing vapor stream and a residue; (b) a mixing vessel that is configured to receive and to combine the cannabinoid-containing vapor stream with a high boiling-point carrier agent to provide a second input mixture, wherein one or more cannabinoids are extracted into the high boiling-point carrier agent in the second input mixture; and (c) a distillation unit for receiving the second input mixture, wherein the distillation unit is configured to separate a first cannabinoid within the second input mixture from at least a second cannabinoid.

The mixing vessel may be any vessel capable of combining the first input mixture or the cannabinoid-containing vapor stream with the high boiling-point carrier agent to provide the second input mixture. The mixing vessel may be of any suitable shape or size, and may for example be of industrial scale or lab scale. Non-limiting examples of mixing vessels include industrial grade mixers, mixing tanks, and mixing drums.

As used herein, the term “volatizing unit” refers to a unit or component that can volatilize or evaporate a substance. The volatizing unit can be of any suitable structure for volatilizing the input mixture. In an embodiment, the volatizing unit may be an evaporator, a wiped film evaporator, a short path distillation unit, a rising-falling film evaporator, a pot still, a jacketed or heated vessel, a centrifugal evaporator, a centrifugal short path distillation, or any combination thereof.

Wiped film evaporation is a method used for separation of thermally-sensitive components. The method provides a short residence time and low evaporation temperature, which help to prevent degradation of one or more thermally sensitive target-components.

In some embodiments of the present disclosure, the volatizing unit is a wiped film evaporator, such as an LCI LabVap® Thin-Film Evaporator that includes a heated surface and a rotating wiping system that physically agitates the input mixture therein for heat and mass transfer of the one or more cannabinoids to a vapor state. In some embodiments, the volatizing unit is a VKL70-5 Shortpath Distillation System for causing a heat and mass transfer of the one or more cannabinoids to a vapor state.

As used herein, the term “distillation unit” refers to a unit or component that can perform a physical separation of compounds (such as cannabinoids) comprised in the cannabinoid-containing vapor stream or second input mixture based on boiling point of the compounds. In an embodiment, the distillation unit may comprise a distillation column. The distillation column may be of any suitable shape or size including, but not limited to, a vertical cylindrical column or a distillation tower. In an embodiment, the distillation unit is a fractional distillation unit. A fractional distillation unit separates a mixture into one or more component parts or fractions. In an embodiment, the distillation unit comprises a temperature control unit and/or a pressure control unit. In an embodiment, the distillation unit can be configured to perform both evaporation and condensation.

In an embodiment, the distillation column defines a plenum that may house packing material, or not. In an embodiment, the plenum is configured to separate the first cannabinoid from the second cannabinoid. In an embodiment, the plenum is configured to separate the first cannabinoid from the second cannabinoid and at least a third cannabinoid. In an embodiment, the plenum is configured to separate the first cannabinoid from the second cannabinoid, the third cannabinoid, and at least a fourth cannabinoid. In an embodiment, the plenum is configured to separate the second cannabinoid from the third cannabinoid and/or the fourth cannabinoid.

As used herein, the term “cannabinoid” refers to: (i) a chemical compound belonging to a class of secondary compounds commonly found in plants of genus cannabis; and/or (ii) one of a class of diverse chemical compounds that may act on cannabinoid receptors such as CB1 and CB2.

In select embodiments of the present disclosure, the cannabinoid is a compound found in a plant, e.g., a plant of genus cannabis, and is sometimes referred to as a phytocannabinoid. One of the most notable cannabinoids of the phytocannabinoids is tetrahydrocannabinol (THC), the primary psychoactive compound in cannabis. Cannabidiol (CBD) is another cannabinoid that is a major constituent of the phytocannabinoids. There are at least 113 different cannabinoids isolated from cannabis, exhibiting varied effects.

In many cases, a cannabinoid can be identified because its chemical name will include the text string “*cannabi*”. However, there are a number of cannabinoids that do not use this nomenclature, such as for example those described herein.

As well, any and all isomeric, enantiomeric, or optically active derivatives are also encompassed. In particular, where appropriate, reference to a particular cannabinoid includes both the “A Form” and the “B Form”. For example, it is known that THCA has two isomers, THCA-A in which the carboxylic acid group is in the 1 position between the hydroxyl group and the carbon chain (A Form) and THCA-B in which the carboxylic acid group is in the 3 position following the carbon chain (B Form).

Examples of cannabinoids include, but are not limited to, Cannabigerolic Acid (CBGA), Cannabigerolic Acid monomethylether (CBGAM), Cannabigerol (CBG), Cannabigerol monomethylether (CBGM), Cannabigerovarinic Acid (CBGVA), Cannabigerovarin (CBGV), Cannabichromenic Acid (CBCA), Cannabichromene (CBC), Cannabichromevarinic Acid (CBCVA), Cannabichromevarin (CBCV), Cannabidiolic Acid (CBDA), Cannabidiol (CBD), Δ6-Cannabidiol (Δ6-CBD), Cannabidiol monomethylether (CBDM), Cannabidiol-C4 (CBD-C4), Cannabidivarinic Acid (CBDVA), Cannabidivarin (CBDV), Cannabidiorcol (CBD-C1), Tetrahydrocannabinolic acid A (THCA-A), Tetrahydrocannabinolic acid B (THCA-B), Tetrahydrocannabinol (THC or Δ9-THC), Δ8-tetrahydrocannabinol (Δ8-THC), trans-Δ10-tetrahydrocannabinol (trans-Δ10-THC), cis-Δ10-tetrahydrocannabinol (cis-Δ10-THC),Tetrahydrocannabinolic acid C4 (THCA-C4), Tetrahydrocannabinol C4 (THC-C4), Tetrahydrocannabivarinic acid (THCVA), Tetrahydrocannabivarin (THCV), Δ8-Tetrahydrocannabivarin (Δ8-THCV), Δ9-Tetrahydrocannabivarin (Δ9-THCV), Tetrahydrocannabiorcolic acid (THCA-C1), Tetrahydrocannabiorcol (THC-C1), Δ7-cis-iso-tetrahydrocannabivarin, Δ8-tetrahydrocannabinolic acid (Δ8-THCA), Δ9-tetrahydrocannabinolic acid (Δ9-THCA), Cannabicyclolic acid (CBLA), Cannabicyclol (CBL), Cannabicyclovarin (CBLV), Cannabielsoic acid A (CBEA-A), Cannabielsoic acid B (CBEA-B), Cannabielsoin (CBE), Cannabinolic acid (CBNA), Cannabinol (CBN), Cannabinol methylether (CBNM), Cannabinol-C4 (CBN-C4), Cannabivarin (CBV), Cannabino-C2 (CBN-C2), Cannabiorcol (CBN-C1), Cannabinodiol (CBND), Cannabinodivarin (CBDV), Cannabitriol (CBT), 11-hydroxy-Δ9-tetrahydrocannabinol (11-OH-THC), 11-nor 9-carboxy-Δ9-tetrahydrocannabinol, Ethoxy-cannabitriolvarin (CBTVE), 10-Ethoxy-9-hydroxy-Δ6a-tetrahydrocannabinol, Cannabitriolvarin (CBTV), 8,9 Dihydroxy-Δ6a(10a)-tetrahydrocannabinol (8,9-Di-OH-CBT-C5), Dehydrocannabifuran (DCBF), Cannbifuran (CBF), Cannabichromanon (CBCN), Cannabicitran, 10-Oxo-Δ6a(10a)-tetrahydrocannabinol (OTHC), Δ9-cis-tetrahydrocannabinol (cis-THC), Cannabiripsol (CBR), 3,4,5,6-tetrahydro-7-hydroxy-alpha-alpha-2-trimethyl-9-n-propyl-2,6-methano-2H-1-benzoxocin-5-methanol (OH-iso-HHCV), Trihydroxy-delta-9-tetrahydrocannabinol (triOH-THC), Yangonin, Epigallocatechin gallate, Dodeca-2E, 4E, 8Z, 10Z-tetraenoic acid isobutylamide, hexahydrocannibinol, and Dodeca-2E,4E-dienoic acid isobutylamide.

As used herein, the term “THC” refers to tetrahydrocannabinol. “THC” is used interchangeably herein with “Δ9-THC”.

Structural formulae of cannabinoids of the present disclosure may include the following:

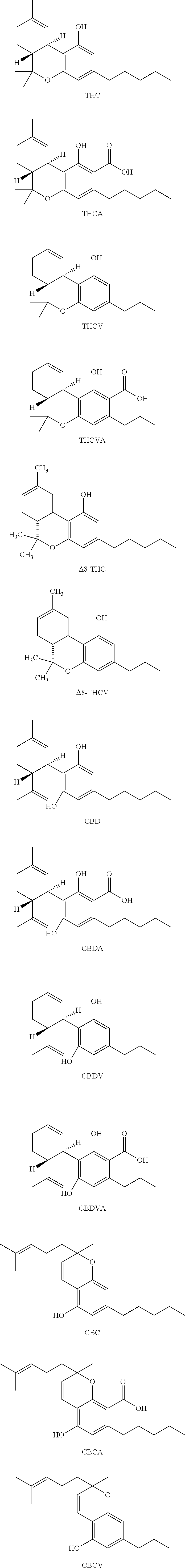

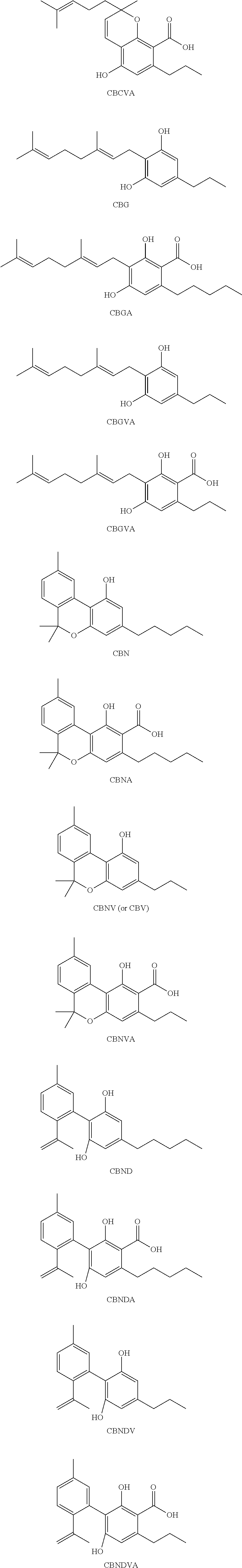

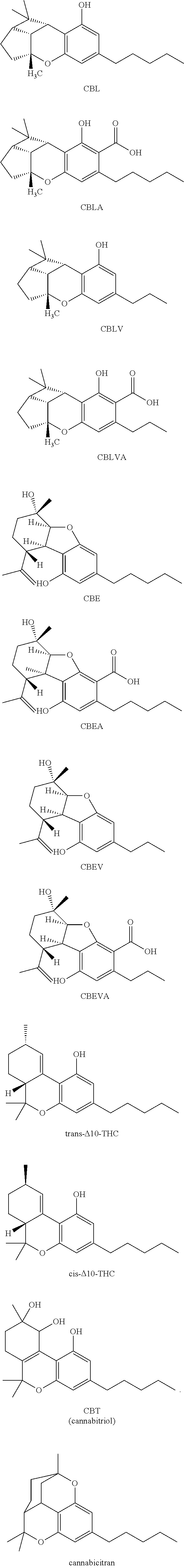

In the context of the present disclosure, the terms “first cannabinoid”, “second cannabinoid”, “third cannabinoid”, “fourth cannabinoid”, and so on, include and encompass any cannabinoid, including any of those described herein.

In select embodiments, the cannabinoid isolated by the apparatus and methods disclosed herein may for example and without limitation be any of those described herein. In a particular, embodiment, the cannabinoid isolated by the apparatus and methods disclosed herein may be THC, Δ8-THC, trans-Δ10-THC, cis-Δ10-THC, THCV, Δ8-THCV, Δ9-THCV, CBD, CBDV, CBC, CBCV, CBG, CBGV, CBN, CBNV, CBND, CBNDV, CBE, CBEV, CBL, CBLV, CBT, or cannabicitran.

In an embodiment, the cannabinoid isolated by the apparatuses and methods disclosed herein may comprise CBD, CBDV, CBC, CBCV, CBG, CBGV, THC, THCV, or a regioisomer thereof. As used herein, the term “regioisomers” refers to compounds that differ only in the location of a particular functional group.

In an embodiment, the isolated cannabinoid is THC.

In an embodiment, the isolated cannabinoid is CBD.

In a particular embodiment, the apparatuses and methods disclosed herein may be employed to provide enrichment of THC relative to CBD, such as for example as shown in Example 11.

As used herein, the term “input mixture” includes any cannabis plant material or extract thereof. Where the apparatuses or methods herein describe a “first input mixture” and a “second input mixture”, it is the first input mixture that is the original source material (i.e. plant material or extract thereof), whereas the subsequent input mixtures (e.g. second or third) are a mixture formed in accordance with the described methods using a first input mixture.

In an embodiment, the input mixture is a cannabis concentrate. As used herein, the term “cannabis concentrate” refers to a mixture of cannabinoids that is obtained from a cannabis plant, such as for example a mixture of compounds or compositions that have been extracted from cannabis. Non-limiting embodiments of a cannabis concentrate include a cannabis distillate, a cannabis isolate, a cannabis resin, or any other type of extract containing one or more cannabinoids or terpenes, or both. In an embodiment, the cannabis concentrate is a cannabis resin.

In embodiments of the apparatuses and methods disclosed herein, a first input mixture may be combined with a high boiling-point carrier agent to provide a second input mixture. In particular embodiments, the second input mixture is comprised of substantially all of the first input mixture and high boiling-point carrier agent. By this, it is meant that none of the first input mixture (e.g. cannabis concentrate or cannabis resin) is removed from the second input mixture, such as by filtration or some other means of separation. Such capability of the apparatus and methods disclosed herein is advantageous at least in respect of efficiency. In an embodiment, the second input mixture comprises all or substantially all of the first input mixture that is used in the apparatuses and methods disclosed herein. In an embodiment, a majority component of the second input mixture is the first input mixture (e.g. cannabis concentrate). By “majority component of the second input mixture”, it is meant that the first input mixture makes up a quantity of the second input mixture of at least 50% or greater, measured on a weight-by-weight or volume-by-volume basis.

As used herein, the term “high boiling-point carrier agent” refers to a hydrophobic substance (e.g. a liquid) into which one or more cannabinoids can be extracted and that has a boiling point (measured under atmospheric conditions) that is higher than the boiling point of at least one or more cannabinoids within the input mixture. For the purposes of this disclosure, the terms “extract”, “extracted” and “extraction” refer to the transfer of one or more chemical compounds, such as one or more cannabinoids, from the input mixture into the high boiling-point carrier agent.

In an embodiment, the high boiling-point carrier agent may be a high smoke-point oil. Exemplary and non-limiting embodiments include avocado oil (refined), almond oil, corn oil, canola oil, grapeseed oil, peanut oil, safflower oil, sesame oil and sunflower oil.

In an embodiment, the high boiling-point carrier agent is a vegetable oil, corn oil, canola oil, safflower oil, avocado oil, mineral oil, silicone oil, quinolone, alkyl derivatives of quinolone, ethylene glycol, glycol ethers, phthalates, triphenylmethane, triphenylmethane derivatives, or any combination thereof. In an embodiment, the carrier agent may be refined, bleached and/or deodorized.

In an embodiment, the high boiling-point carrier agent has a boiling point that is at least about 5° C. to 150° C. higher than the boiling point of at least one cannabinoid within the input mixture (measured under atmospheric pressure conditions), such as for example the cannabinoid that is desired to be isolated. In an embodiment, the high boiling-point carrier agent has a boiling point that is at least about 15° C. to 125° C. higher than the boiling point of at least one cannabinoid within the input mixture. In an embodiment, the high boiling-point carrier agent has a boiling point that is at least about 25° C. to 100° C. higher than the boiling point of at least one cannabinoid within the input mixture. In a particular embodiment, the high boiling-point carrier agent has a boiling point that is at least 50° C. higher than a boiling point of at least one cannabinoid within the input mixture.

In the context of the present disclosure, the term “residue” can refer to either an amount of input mixture that is not dissolved in the high boiling-point carrier agent or to the input mixture remaining in the volatizing unit after volatilizing to provide the cannabinoid-containing vapor stream.

In another aspect, the present disclosure relates to methods of isolating one or more cannabinoids from an input mixture.

In an embodiment, the present disclosure relates to a method of isolating one or more cannabinoids from an input mixture, the method comprising the steps of: (a) combining a first input mixture with a high boiling-point carrier agent to provide a second input mixture, wherein one or more cannabinoids from the first input mixture are extracted into the high boiling-point carrier agent in the second input mixture; (b) volatilizing the second input mixture to provide a cannabinoid-containing vapor stream and a residue; (c) conducting the cannabinoid-containing vapor stream to a distillation unit that separates a first cannabinoid within the cannabinoid-containing vapor stream from at least a second cannabinoid; and (d) collecting a product comprising the first cannabinoid.

In an embodiment, the present disclosure relates to a method of isolating one or more cannabinoids from an input mixture, the method comprising steps of: (a) introducing a first input mixture into a volatizing unit for separating the first input mixture into a cannabinoid-containing vapor stream and a residue; (b) combining the cannabinoid-containing vapor stream with a high boiling-point carrier agent to provide a second input mixture, wherein one or more cannabinoids from the cannabinoid-containing vapor stream are extracted into the high boiling-point carrier agent in the second input mixture; (c) conducting the second input mixture to a distillation unit that separates a first cannabinoid within the second input mixture from at least a second cannabinoid; and (d) collecting a product that comprises the first cannabinoid.

The combining may be done by any suitable means to dissolve at least one cannabinoid in the first input mixture into the high boiling-point carrier agent. In an embodiment, the combining is by the mixing unit described elsewhere herein. In an embodiment, the combining is by adding, mixing or shaking. In an embodiment, the combining is by manual mixing. The combining may be performed over any period of time suitable to extract at least one cannabinoid into the high boiling-point carrier agent.

The volatilizing may be done by any suitable means to cause at least one cannabinoid in the input mixture to vaporize. In an embodiment, the volatilizing can be by an evaporator, a wiped film evaporator, a short path distillation unit, a rising-falling film evaporator, a pot still, a jacketed or heated vessel, a centrifugal evaporator, a centrifugal short path distillation or combinations thereof. In a particular embodiment, the volatilizing is by a wiped film evaporator as described elsewhere herein.

The conducting may be by any suitable means including, but not limited to, passing the input mixture through a conduit. Non-limiting examples of conduits include tubing and piping. The conduit may be of any suitable size and material.

The collecting may be done by any suitable means to obtain all or a portion of a product containing the cannabinoid. In an embodiment, the collecting of the product that comprises a first cannabinoid is by one or more distillation trays. A distillation tray may facilitate condensation of one or more cannabinoids. In an embodiment, the collecting is by a suitable container, such as a round-bottom flask or a collection flask.

For use in the methods disclosed herein, the high boiling-point carrier agent is as described elsewhere herein. For example, in an embodiment of the methods herein, the high boiling-point carrier agent is a vegetable oil, corn oil, canola oil, safflower oil, avocado oil, mineral oil, silicone oil, quinolone, alkyl derivatives of quinolone, ethylene glycol, glycol ethers, phthalates, triphenylmethane, triphenylmethane derivatives, or any combination thereof. In an embodiment, the high boiling-point carrier agent may be refined, bleached and/or deodorized. In a particular embodiment, the high boiling-point carrier agent is canola oil or avocado oil.

In the context of the present disclosure, the quantity of a cannabinoid in a particular composition may be expressed in various fashions, such as but not limited to a percentage of the total weight of the particular composition or as a ratio that represents the relative quantity of a cannabinoid as compared to another compound within the particular composition. Additionally, the relative quantities of two cannabinoids (for example a first cannabinoid relative to a second cannabinoid) in a particular composition may be expressed as a ratio (for example first cannabinoid:second cannabinoid). The relative quantities of cannabinoid products in a mixture may be referred to with analogous ratios (e.g. second cannabinoid:third cannabinoid). Those skilled in the art will recognize that a variety of analytical methods may be used to determine such quantities and ratios, and the protocols required to implement any such method are within the purview of those skilled in the art. By way of non-limiting example, such ratios may be determined by diode-array-detector high pressure liquid chromatography, UV-detector high pressure liquid chromatography, nuclear magnetic resonance spectroscopy, mass spectroscopy, flame-ionization gas chromatography, gas chromatograph-mass spectroscopy, or any combination thereof.

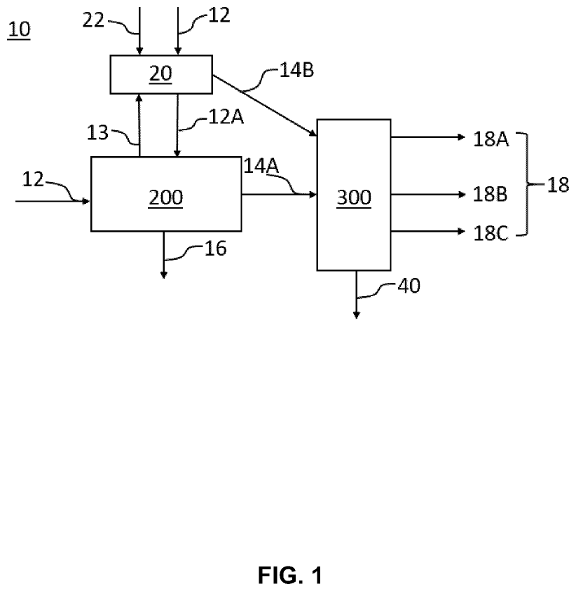

Embodiments of the present disclosure will now be described in detail including references to FIG. 1 to FIG. 5, which show embodiments of the apparatus and methods according to the present disclosure.

FIG. 1 shows a non-limiting example of an apparatus 10 that comprises a mixing vessel 20, a volatizing unit 200 (e.g. a wiped film evaporator), and a distillation unit 300 (which may also be referred to as a rectifier). In this embodiment, the mixing vessel 20 may be configured to combine the first input mixture 12 with a high boiling-point carrier agent 22 to provide a second input mixture 12A, wherein one or more cannabinoids in the first input mixture 12 are extracted into the high boiling-point carrier agent 22 within the second input mixture 12A. The high boiling-point carrier agent 22 may be any of those described elsewhere herein.

The second input mixture 12A comprises the one or more cannabinoids extracted within the high boiling-point carrier agent 22 and potentially other non-extracted constituents of the first input mixture 12.

The high boiling-point carrier agent 22 may be added to the first input mixture 12 to form the second input mixture 12A. The second input mixture 12A may be an extract of the mixture of the first input mixture 12 and the high boiling-point carrier agent 22, or not. In an embodiment, the first input mixture 12 is a cannabis concentrate or cannabis resin and is combined with the high boiling-point carrier agent 22 to provide the second input mixture 12A which is a mixture of both, wherein substantially all of the first input mixture 12 and the high boiling-point carrier agent 22 are contained within the second input mixture 12A. In the second input mixture 12A, at least one or more cannabinoids are extracted into the high boiling-point carrier 22 component of the second input mixture 12A.

The volatizing unit 200 (e.g. a wiped film evaporator) may be configured to receive and volatilize the second input mixture 12A to produce a cannabinoid-containing vapor stream 14A and a residue 16. For example, the volatizing unit 200 may apply heat and physical agitation to the second input mixture 12A to provide the cannabinoid-containing vapor stream 14A and the residue 16. It will be appreciated by those skilled in the art that the cannabinoid-containing vapor stream 14A may comprise further constituents of the first input mixture 12. In some embodiments of the present disclosure, substantially most, substantially all, or all of the cannabinoids within the second input mixture 12A are volatilized and entrained in the cannabinoid-containing vapor stream 14A. The residue 16 may be substantially free of cannabinoids, or not. In some embodiments, the residue 16 may be conducted back (not shown) to join either or both of the first input mixture 12 and the second input mixture 12A. In an embodiment, the volatizing unit 200 is an LCI LabVap® Thin-Film Evaporator that includes a heated surface and a rotating wiping system that physically agitates the input mixture 12A therein for heat and mass transfer of the one or more cannabinoids to a vapor state. In an embodiment, the volatizing unit 200 is a VKL70-5 wiped film shortpath distillation system for heat and mass transfer of the one or more cannabinoids to a vapor state.

As will be appreciated by the skilled reader, within the volatizing unit 200, the second input mixture 12A can be heated on an internal surface of a heated tube (not shown). A rotating wiping system (not shown) inside the heated tube can generate a very thin and turbulent film on the surface of the heated tube. The turbulent film can optimize heat and mass transfer to volatilize at least a portion of the second input mixture 12A to produce the cannabinoid-containing vapor stream 14A and the residue 16.

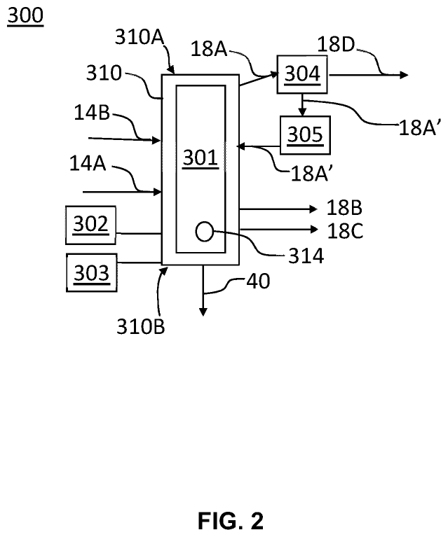

In some embodiments, the cannabinoid-containing vapor stream 14A can be conducted, for example through one or more fluid conduits, to the distillation unit 300 for separating one or more cannabinoids from the cannabinoid-containing vapor stream 14A. The distillation unit 300 can be configured to separate heat-sensitive compounds within the input mixture where such compounds have a relatively narrow boiling-point difference for producing at least one cannabinoid output stream 18, which may also be referred to as a distillate or product, and a waste stream 40. In some embodiments, the distillation unit 300 can be a fractional distillation unit that is configured to separate more than one cannabinoid output steam, such as a first cannabinoid output stream 18A, a second cannabinoid output stream 18B, a third cannabinoid output stream 18C from the other constituents within the cannabinoid-containing vapor stream 14A. The at least one cannabinoid output stream 18 may comprise more or less output streams than are shown in FIG. 1. In an embodiment, the distillation unit 300 is a continuous fractional distillation unit that continuously separates the at least one cannabinoid output stream 18 from the other constituents within the cannabinoid-containing vapor stream 14A. In an embodiment, the waste stream 40 may be conducted back (not shown) to join the first input mixture 12 or the second input mixture 14A, as described further below. The waste stream 40 may be collected from the second end 310B (FIG. 2).

In some embodiments, volatizing unit 200 may be configured to receive and volatilize the first input mixture 12 to provide a cannabinoid-containing vapor stream 13. The cannabinoid-containing vapor stream 13 can be conducted, for example through one or more fluid conduits, to the mixing vessel 20. The mixing vessel 20 may be configured to combine the cannabinoid-containing vapor stream 13 with the high boiling-point carrier agent 22 to generate a second input mixture 14B, wherein at least one cannabinoid from the cannabinoid-containing vapor stream 13 can be extracted into the high boiling-point carrier agent 22 within the second input mixture 14B. The second input mixture 14B, comprising at least one cannabinoid, can be conducted, for example through one or more fluid conduits, to the distillation unit 300.

As shown in FIG. 2, the distillation unit 300 may comprise a distillation column 310 with a first end 310A, which may also be referred to as the column head, and a second end 310B. Together these two ends 310A, 310B define an internal plenum 301. The plenum 301 can house one or more separation trays (not shown) that are configured to separate at least one cannabinoid from within the cannabinoid-containing vapor stream 14A, the second input mixture 14B, or a combination of both from other constituents of these inputs which may include at least a second cannabinoid, a third cannabinoid, a fourth cannabinoid and further cannabinoids.

In some embodiments, distillation unit 300 may comprise both a temperature control unit 302 and pressure control unit 303. The temperature control unit 302 can be configured to heat or cool the temperature within the plenum 301. In an embodiment, the column 310 may be sealed at each end so that the plenum is isolated from the ambient pressure and the pressure control unit 303 is configured to increase or decrease the pressure within the plenum 301. In an embodiment, the distillation unit is a continuous fractional distillation unit 300 that operates at a temperature of between about 100° C. and about 275° C. or between about 120° C. and about 150° C. within a range of pressures of between about 0.001 mbar and 110 mbar.

In some embodiments, the plenum 301 may house a packing material 314. The packing material 314 may include, but is not limited to, the following: MONTZ-Pak Type A3-750, MONTZ-Pak Type A3-1000, MONTZ-Pak Type A3-1200, MONTZ-Pak Type A3-1500, MONTZ-Pak Type A3-1900, Sulzer laboratory packing DX, or any combination thereof.

In some embodiments, the distillation unit 300 may further comprise a condenser 304 that is operatively connected at, near, or to the first end 310A. The condenser 304 can be configured to condense a lighter vaporized cannabinoid fraction (for example the first cannabinoid output stream 18A, which may be the most volatile cannabinoid within the mixture cannabinoids) to form a condensed first cannabinoid output stream 18D. Optionally, a portion of the condensed distillate 18A′ can be conducted from the condenser 304 to one or more distributors 305. The distributors 305 may be configured to conduct at least part of the portion of the condensed steam 18A′ back into the plenum 301 where the portion of the condensed stream 18A′ can mix with the cannabinoid-containing stream 14A or the second input mixture 14B within the distillation unit 300. The second cannabinoid-containing output stream (product) 18B and potentially the third cannabinoid-containing output stream (product) 18C (and potentially others) can also be collected from the distillation unit 300.

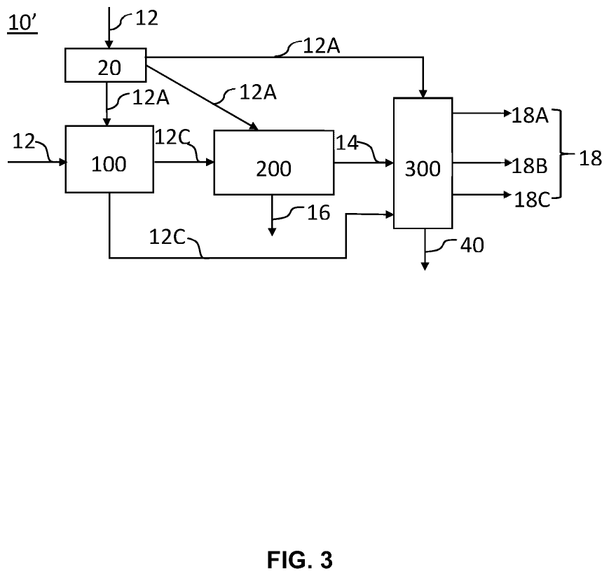

As shown in the non-limiting example of FIG. 3, some embodiments of the present disclosure relate to an apparatus 10′ that comprises a processing unit 100 that is configured to process the first input mixture 12, the second input mixture 12A or any combination thereof for producing a third input mixture 12C. The apparatus 10′ has many of the same components as the apparatus 10, with at least one difference being the processing unit 100. The third input mixture 12C may be conducted to the volatizing unit 200, the distillation unit 300 or any combination thereof as discussed herein above. In some embodiments of the present disclosure, the second input mixture 12A may be conducted to the processing unit 100, to the volatizing unit 200, the distillation unit 300 or any combination thereof. In some embodiments, the first input mixture 12 may be introduced to processing unit 100, and in such embodiments the high-boiling carrier agent 22 may be introduced during processing in the processing unit 100 or the processed first input mixture may be conducted to the mixing vessel 20 (step not shown).

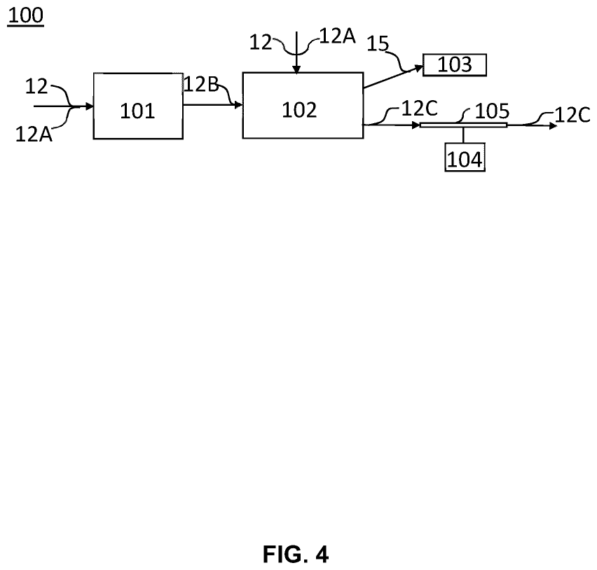

As shown in the non-limiting example of FIG. 4, in an embodiment, the processing unit 100 comprises a heatable tank 101, a degassing chamber 102, and a condenser 103. The heatable tank 101 can be configured to receive the first input mixture 12, the second input mixture 12A or a combination thereof. In some embodiments, the first input mixture 12 is introduced to the heatable tank 101, and in such embodiments the processing unit 100 may comprise a means of introducing the high boiling-point carrier agent 22. In some embodiments, the heatable tank 101 may be heated and, optionally, under vacuum for providing a heated input mixture 12B. Heated input mixture 12B can be heated first input mixture 12, heated second input mixture 12A, or a heated combination of both. The heated input mixture 12B may be conducted to the degassing chamber 102 that may also be operated under vacuum. In an embodiment, the first input mixture 12, the second input mixture 12A or a combination thereof may be introduced to the degassing chamber 102. In some embodiments, the first input mixture 12 is introduced to the degassing chamber 102, and in such embodiments the processing unit 100 may comprise a means of introducing the high boiling-point carrier agent 22. In some embodiments, the condenser 103 may be used to condense one or more gaseous compounds 15 that are removed by the degassing chamber 102. The one or more gaseous compounds 13 can be condensable or non-condensable gases. Some examples of condensable gases include, but are not limited to, solvent residue (ethanol, butane and the like), volatile organic compounds, such as terpenes and water. In an embodiment, the primary non-condensable gas is CO₂. In an embodiment, the processing unit 100 may also comprise a transfer pump 104 and heated hose 105 that are configured to conduct the third input mixture 12C either to the first end 310A of the distillation column 310, the middle of the distillation column 310, the mixing vessel 20, the volatizing unit 200, the distillation unit 300, or any combination thereof (FIG. 3).

In some embodiments of the present disclosure, there is no distillation unit 300 and the volatilization of the second input mixture 12A provides sufficient separation of the one or more desired cannabinoids from the first input mixture 12.

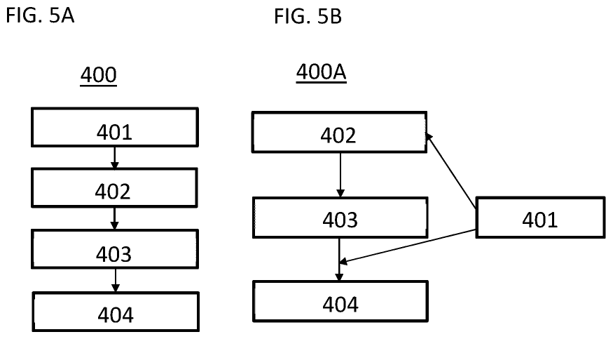

FIG. 5A shows a sequence of steps that form part of a first method 400 according to embodiments of the present disclosure that relates to isolating one or more cannabinoids from an input mixture by using a high boiling-point carrier agent and distillation.

The first method 400 comprises a step 401 of combining the first input mixture 12 with a high boiling-point carrier agent to provide a second input mixture 12A, wherein one or more cannabinoids from the first input mixture 12 are extracted into the high boiling-point carrier agent.

Step 402 comprises volatilizing the second input mixture to provide a cannabinoid-containing vapor stream and a residue.

The method 400 further comprises a step 403 of conducting the cannabinoid-containing vapor stream to a distillation unit that separates a first cannabinoid within the cannabinoid-containing vapor stream from at least a second cannabinoid. In some embodiments of the present disclosure, the step 403 can be achieved by conducting the cannabinoid-containing vapor stream 14A to a distillation unit 300 and through the distillation column 310. In some embodiments of the present disclosure, the distillation unit 300 may be a fractional distillation unit that operates continuously or discontinuously. The distillation unit 300 may be operated at specific temperatures and pressures while performing the step 403 within the distillation unit 300.

The method 400 further comprises a step 404 of collecting a product comprising the first cannabinoid.

FIG. 5B shows another method 400A according to embodiments of the present disclosure that includes many of the same steps as method 400 described above. At least one difference between method 400 and method 400A is that the step of making the second input mixture 12A can occur after the step 403.

### EXAMPLES

### Example 1

### Input Mixture

The input mixture containing cannabinoids may be a cannabis resin that was prepared by first preparing a precursor extract by the steps of dissolution, chilling and filtering to produce the precursor extract, which may be substantially transparent. Optionally, the precursor extract can be substantially or completely depleted of lipids and waxes. The precursor extract can have a cannabinoid concentration of between about 60% and about 90% (wt/wt) or between about 70% and about 80% (wt/wt).

Next, the cannabis resin was combined with a high boiling-point carrier agent in a mixing vessel, providing an input mixture, wherein at least a first cannabinoid was extracted into the carrier agent. The high boiling-point carrier agent may include, but is not limited to: vegetable oil, corn oil, canola oil, safflower oil, avocado oil, mineral oil; silicone oil; quinolone and its alkyl derivatives; ethylene glycol; glycol ethers; phthalates; triphenylmethane, triphenylmethane derivatives; or, any combination thereof.

The second input-mixture was introduced into a wiped film evaporator. The cannabinoids were evaporated from the mixture into cannabinoid-containing vapor stream inside the wiped film evaporator and were conducted in the vapor phase to an external continuous distillation unit. Non-volatile components (e.g. residue) were discharged from the bottom of the wiped film evaporator.

### Example 2

The cannabinoid-containing vapor stream was conducted through the fractional distillation column where at least one cannabinoid in the cannabinoid-containing vapor stream was separated from the other constituents of the cannabinoid-containing vapor stream. The product stream was collected from the distillation unit and non-volatile components were discharged from the bottom of the distillation unit.

### Example 3

The input mixture of Example 1 was introduced into the wiped film evaporator. The cannabinoids were evaporated from the input mixture into a cannabinoid-containing vapor stream inside the wiped film evaporator and were conducted in the vapor phase to the mixing vessel. The mixing vessel was configured to combine at least one carrier agent with the cannabinoid-containing vapor stream to provide a second input mixture. The second input mixture, comprising at least one cannabinoid extracted from the cannabinoid-containing vapor stream, was conducted to the distillation unit.

The second input mixture was exposed to heat and pressure in the distillation unit and the cannabinoid-containing vapor stream was conducted through a fractional distillation column where at least one cannabinoid in the cannabinoid-containing vapor stream was separated from the other constituents of the cannabinoid-containing vapor stream. A product was collected from the distillation unit and non-volatile components were discharged from the distillation unit.

### Example 4

### Short Path Distillation

In instances where the input mixture comprises volatile organic compounds (VOCs) and/or acidic cannabinoids, the input mixture may not be suitable for direct input into a short path distillation unit as the volatizing unit. This is because the VOCs can off gas and interfere with the vacuum pressure of the short path distillation unit. Additionally, the acidic cannabinoids can decarboxylate at the distillation temperatures contemplated herein, which generates CO₂ that can also interfere with the vacuum pressure of the short path distillation unit. Instead, this type of input mixture can be subjected to a two-staged process that includes a first degassing step and a second short path distillation step and then subjected to the fractional distillation process.

The first step includes degassing by heating these types of input mixtures to about 100° C. in a feed hopper and passing the heated input mixture through a 0.25 cubic foot horizontal wipe-film evaporator that is operating at a temperature of about 150° C. and at feed speed of about 150 mL per minute. The vacuum is maintained at about 0.5 atm absolute with appropriately sized condenser set to about 4° C. to collect any volatile compounds that are evaporated. This process effectively strips the input mixture of some or substantially all volatile compounds or acidic cannabinoids that may interfere later with short path distillation. Alternatively, the input mixture may be heated on a hotplate to about 150° C. with stirring until no more gas is produced.

After this degassing, the input mixture is maintained at about 100° C. and pumped into the shortpath distillation system where it is heated to between about 120° C. and about 250° C.—depending on the operating pressure—causing the cannabinoids to evaporate. The evaporated cannabinoids can be directed to the fractional distillation unit or they may be re condensed on an internal condenser set at about 50° C. and about 60° C. and any remaining low boiling point compounds are collected using a cold trap that is at about −40° C. ahead of the vacuum pump.

### Example 5

### Short Path Distillation for Enrichment of CBD at Various Temperatures

An input mixture was prepared using a plant input that had on average 14 wt/wt % CBD and <1 wt/wt % THC. The input mixture was subjected to a two-step shortpath distillation process (using the VKL70-5 Short path distillation system), as described herein above and then subjected to a distillation process at temperatures ranging between about 129° C. and 169° C. The cannabinoid concentration for the residue product (RP) and the distillation product (DP) are provided below in Table 1.

TABLE 1
Cannabinoid concentration of short path distilled
and further distilled input mixture.

| | Cannabinoid Concentration | | | | | | |
|---|---|---|---|---|---|---|---|
| | | | | | | | Residue |
| Temperature | RP | RP | RP Total | DP | DP | DP Total | Ratio: |
| (° C.) | CBD | THC | Cannabinoids | CBD | THC | Cannabinoids | CBD/THC |
| 169 | 3.39 | 0.38 | 3.77 | 67.72 | 2.67 | 75.15 | 8.92 |
| 164 | 2.53 | 2.49 | 82.15 | 82.15 | 2.95 | 89.87 | 14.32 |
| 159 | 3.64 | 3.64 | 81.33 | 81.33 | 2.58 | 88.56 | 12.74 |
| 154 | 4.52 | 4.52 | 82.27 | 82.27 | 2.42 | 88.53 | 13.76 |
| 149 | 3.06 | 3.06 | 83.28 | 83.28 | 2.28 | 89.48 | 27.80 |
| 144 | 3.21 | 3.21 | 88.33 | 88.33 | 2.03 | 94.42 | 28.45 |
| 139 | 2.59 | 2.59 | 82.81 | 82.81 | 1.41 | 87.5 | 33.97 |
| 134 | 1.75 | 1.75 | 81.87 | 81.87 | 0.95 | 85.8 | 49.33 |
| 129 | 1.33 | 1.33 | 78.62 | 78.62 | 0.92 | 82.58 | 68.60 |

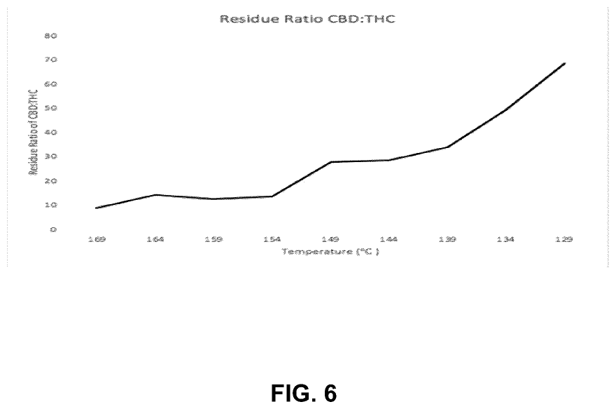

FIG. 6 shows a line graph that represents the RP ratio of CBD:THC plotted over the various distillation temperatures. Without being bound by any particular theory, THC can be distilled out to produce a higher concentration of CBD in a RP as shown by an increasing the ratio of CBD:THC to about 8× when temperatures are at about 129° C. as compared to higher temperatures.

### Example 6

### Short Path Distillation for Enrichment of CBD at Various Pressures

In this example, a second pass distillate was passed through a short path distillation unit (as described above in Example 6) and then subjected to the fractional distillation process at various pressures (2.6-3.0 mbar) and various temperatures (about 169° C. to about 234° C.) in order to assess the evaporation points and the purity of the resulting distillate and residue. Table 2 summarizes the residue/distillate accumulation over the various temperatures utilized.

TABLE 2
Residue and Distillate Accumulation Data

Residue/Distillate Accumulation

| | Input | Residue | Distillate | Distillate/ |
|---|---|---|---|---|
| Temperature | Weight | Weight | Weight | Residue |
| (° C.) | (g) | (g) | (g) | Split |
| 169 | 269.4 | 230.0 | 0 | 0 |
| 179 | 230.0 | 218.1 | 5.0 | 0.02 |
| 189 | 222.5 | 199.3 | 22.8 | 0.11 |
| 194 | 218.8 | 189.3 | 27.9 | 0.15 |
| 199 | 216.0 | 176.1 | 41.8 | 0.26 |
| 204 | 219.6 | 166.6 | 47.3 | 0.28 |
| 209 | 213.9 | 149.3 | 67.1 | 0.44 |
| 214 | 216.3 | 106.6 | 107.8 | 1.01 |
| 219 | 211.8 | 97.5 | 116.5 | 1.19 |
| 224 | 210.6 | 47.1 | 162.9 | 3.45 |
| 229* | 206.2 | 50.8 | 153.1 | 3.01 |
| 229 | 202.8 | 22.4 | 143.7 | 6.42 |
| 234* | 162.4 | 45.6 | 81.5 | 1.79 |
| 234 | 123.4 | 7.7 | 105.0 | 13.63 |

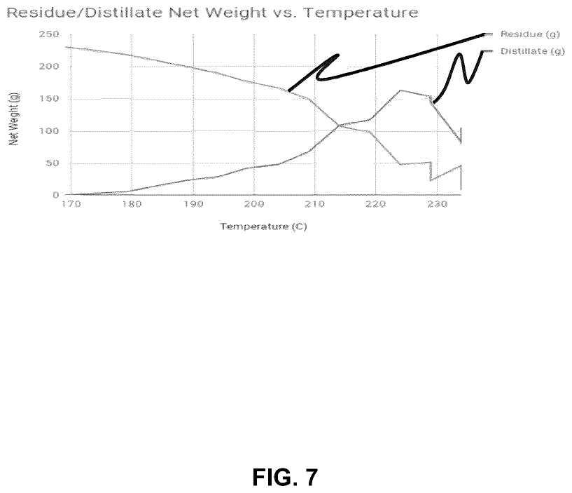

FIG. 7 shows a line graph that represents the net weight versus the temperature of the residue and the distillate, respectively.

Without being bound by any particular theory, the ratio of distillate to residue increased dramatically at about 214° C. This suggests that this may represent an optimal temperature point for evaporation of cannabinoids at 2.6-3 mbar. Higher temperatures were seen to cause burning of material on the inside of the distillation chamber.

### Example 7

### Constant temperature and Constant Feed Rate

An input mixture was subjected to similar experimental conditions as described above in Example 6, but the distillation process was conducted at a constant temperature of 134° C. at a feed rate of 15 Hz. Table 3 summarizes the cannabinoid concentration of the RP and the DP.

TABLE 3
Cannabinoid Concentration of Residue Product and Distillate Product.

| | | Cannabinoid Concentration | | | | | | | |
|---|---|---|---|---|---|---|---|---|---|
| Trial | Temperature | RP | RP | RP Total | DP | DP | DP Total | Ratio: | |
| # | (° C.) | CBD | THC | Cannabinoids | CBD | THC | Cannabinoids | CBD/THC | Enrichment |
| 1 | 134 | 28.43 | 23.70 | 57.61 | 50.01 | 20.97 | 75.94 | 2.38 | 1.41 |
| 2 | 134 | 44.84 | 31.71 | 82.61 | 57.63 | 18.19 | 80.20 | 3.16 | 1.33 |
| 3 | 134 | 63.14 | 19.79 | 87.33 | 65.87 | 16.68 | 86.56 | 3.95 | 1.25 |
| 4 | 134 | 62.56 | 24.35 | 91.76 | 71.44 | 14.55 | 89.63 | 4.91 | 1.24 |

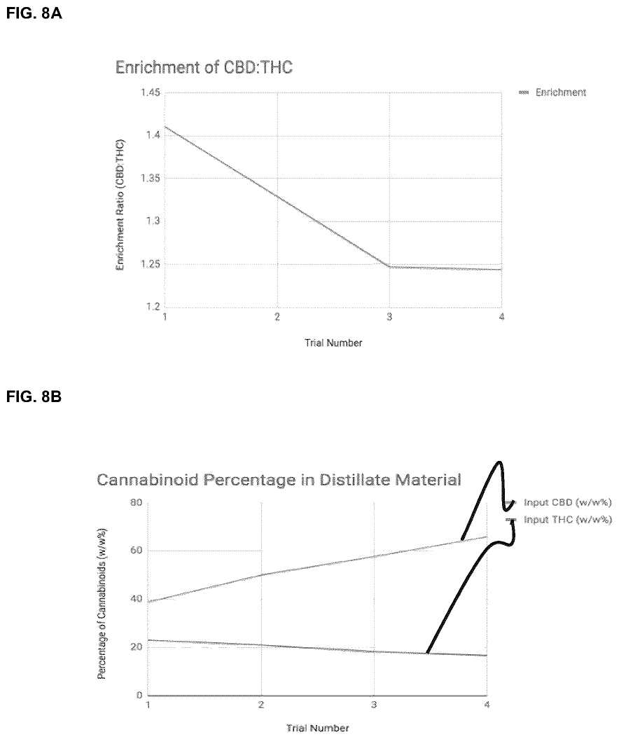

FIG. 8A shows a line graph that represents the enrichment of CBD:THC over the four trials. FIG. 8B shows a line graph that represents the percentage of CBD (upper line) and THC (lower line) in the distillate product.

Without being bound by any particular theory, these results indicate that CBD can be enriched in the distillate product at constant temperature and pressure.

### Example 8

### Variable Feed Rates

An input mixture was subjected to similar experimental conditions as described above in Example 6, but the distillation process was conducted at a constant temperature of 209° C. at a feed rate of between about 2.5 Hz and about 10 Hz and at pressure of between about 8 mmHg and 10 mmHg. Table 4 summarizes the residue/distillate accumulation over the various temperatures utilized.

TABLE 4
Residue and Distillate Accumulation Data

| | Residue/Distillate Accumulation | | | | |
|---|---|---|---|---|---|
| | Input Net | Residue | Distillate | Output Net | Distillate/ |
| Pressure | Weight | Weight | Weight | Weight | Residue |
| (mmHg) | (g) | (g) | (g) | (g) | Split |
| 2 | 213.9 | 149.3 | 67.1 | 216.4 | 0.45 |
| 8 | 610.3 | 411.4 | 194.2 | 605.6 | 0.47 |
| 10 | 596.5 | 379.6 | 187.1 | 566.7 | 0.49 |

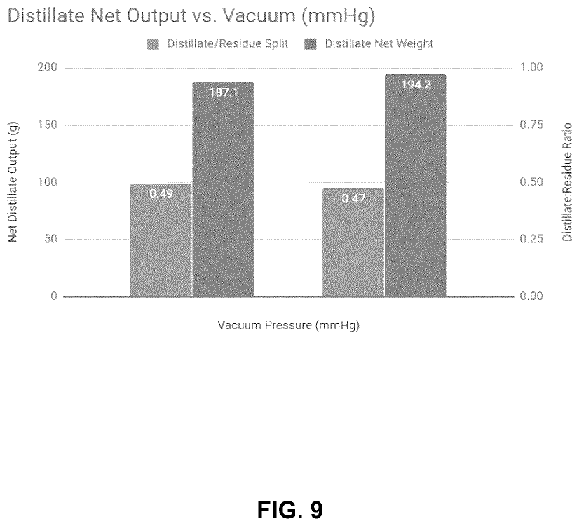

FIG. 9 shows a histogram that represents the distillate net output compared with the pressure within the distillation unit.

Without being bound by any particular theory, the ratio of distillate to residue at 210° C. with a feed rate of 10 Hz remained fairly constant even when the feed rate was decreased to 8 mmHg. Also when compared to 209° C. at 2 mmg with a feed rate of 25 Hz, the spit ratio remained substantially constant.

Table 5 shows the accumulation of residue product and distillate produce at various feed rates (2.5 Hz, 5 Hz and 10 Hz).

TABLE 5
Residue and Distillate Accumulation Data

| | Residue/Distillate Accumulation | | | | |
|---|---|---|---|---|---|
| Feed | Input Net | Residue | Distillate | Output Net | Distillate/ |
| Rate | Weight | Weight | Weight | Weight | Residue |
| (Hz) | (g) | (g) | (g) | (g) | Split |
| 10 | 676.2 | 589.3 | 59.0 | 648.3 | 0.10 |
| 5.0 | 610.3 | 411.4 | 194.2 | 605.6 | 0.47 |
| 2.5 | 314.5 | 28.9 | 276.1 | 305.0 | 9.55 |

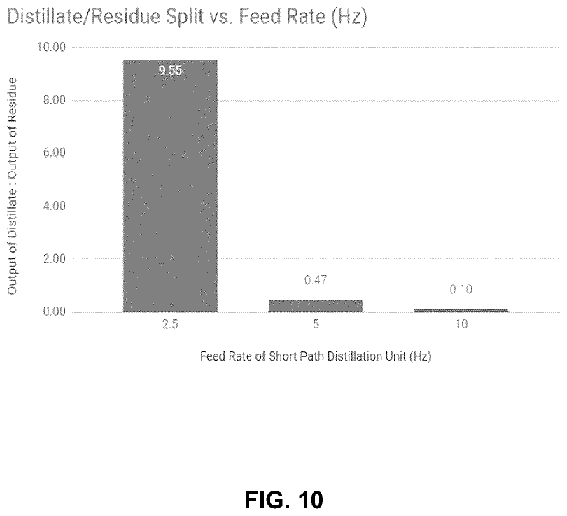

FIG. 10 shows a histogram that represents the distillate/residue split as compared to the feed rate. When using a substantially constant temperature of about 210° C. and a vacuum pressure of about 10 mmHg (13.2-13.4 mbar) for the Short Path Distillation of a Second pass resin, it was found that the most efficient feed speed to promote the greatest evaporation and distillation is about 2.5 Hz. At 2.5 Hz, the material distills over at a distillate to residue ratio of 9.55. Without being bound by any particular theory, this data demonstrates that there may be a significant time to allow for evaporation and distillation of the material. Further, the other parameters (210° C. and 10 mmHg) still promote evaporation within the evaporation chamber without degrading the cannabinoids.

### Example 9

### Omega 3 Oil

Instead of the input mixture, a mixture of omega 3 oils was subjected to a similar set of experimental conditions as Example 6. There were variable temperatures, a substantially constant pressure and a feed rate of about 89 mL/hr. Table 6 shows the accumulation of residue product and distillate product at various temperatures.

TABLE 6
Residue and Distillate Accumulation Data

| | Residue/Distillate Accumulation | | | | | |
|---|---|---|---|---|---|---|
| | Input Net | Residue | Distillate | Output Net | Distillate/ | Percent |
| Temperature | Weight | Weight | Weight | Weight | Residue | Distillate |
| (° C.) | (g) | (g) | (g) | (g) | Split | (%) |
| 160 | 136 | 83 | 22.2 | 105.2 | 0.27 | 21.1 |
| 165 | 74.7 | 38.6 | 32.4 | 71.0 | 0.84 | 45.6 |
| 170 | 76.1 | 28.6 | 46.3 | 74.9 | 1.62 | 61.8 |
| 175 | 44.4 | 3.3 | 38.2 | 41.6 | 11.61 | 93.3 |

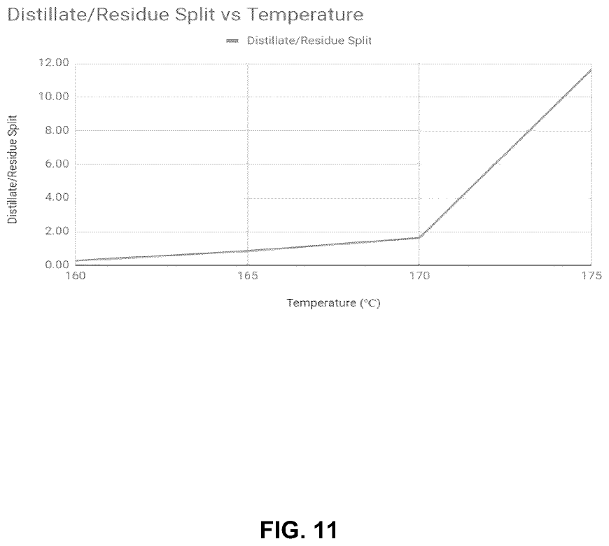

FIG. 11 shows a line graph that represents the distillate/residue split as compared to the various temperatures tested. It was found that at a temperature of about 175° C., over 93% of the Omega-3 Oil INPUT was distilled. A temperature of about 175° C., at a feed speed of about 2.5 Hz and pressure of 10 mmHg produced a distillate to residue ratio of 11.61.

### Example 10

### Parameter Impact in Single Step Short Path Distillation

For instances where the input mixture is substantially free from or has a low content of VOCs and/or acidic cannabinoids, the input mixture may be suitable for direct input into a short path distillation unit and a two-staged process may not be necessary.

Inputs of cannabis resin or hemp resin prepared by CO₂ extraction were subjected to short path distillation at the indicated operating conditions (Table 7) and at an evaporation temperature of 169° C. Table 7 summarizes the input ratio, output ratio and enrichment under the operating conditions. Without being bound by any particular theory, the ratio of CBD:THC in the final distillate product remained similar to that of the input. It was observed that going from 0.035 mbar to 0.008 mbar caused the ratio of CBD:THC to increase from the input material. It was observed that at 169° C. the ratio of CBD:THC remains roughly constant throughout distillation.

TABLE 7
CBD:THC Enrichment under Standard Short Path Distillation Conditions

| | | | | | | Enrichment |
|---|---|---|---|---|---|---|
| | Temperature | Feed Speed | Vacuum | Input Ratio | Output Ratio | Ratio |
| Material | (C.) | (HZ) | (mbar) | CBD:THC | CBD:THC | CBD:THC |
| Cannabis Resin | 169 | 35 | 0.025 | 20.4 | 19.79 | 0.97 |
| Cannabis Resin | 169 | 40 | 0.035 | 0.007 | 0.006 | 0.86 |
| Hemp Resin | 169 | 40 | 0.008 | 23.5 | 25.3 | 1.08 |

Input Ratio CBD:THC = value determined by dividing % w/w CBD in input material by % w/w THC
Output Ratio CBD:THC = value determined by dividing % w/w CBD in output material by % w/w THC
Enrichment Ratio CBD:THC = value determined by dividing the output ratio CBD:THC by the Input Ratio CBD:THC

An input of cannabis resin was subjected to varying temperatures with consistent feed speed and vacuum. Without being bound by any particular theory, as distillation temperature increased, the ratio of CBD:THC in the final distillate product also increased. At approximately 130° C. the ratio of CBD:THC increased in the distillate. Table 8 summarizes the enrichment results.

TABLE 8
Impact of Temperature on CBD:THC Enrichment Under Low Pressure Conditions

| | Temperature | Feed Speed | Vacuum | Input Ratio | Output Ratio | Enrichment Ratio |
|---|---|---|---|---|---|---|
| Material | (C.) | (Hz) | (mbar) | CBD:THC | CBD:THC | CBD:THC |
| Cannabis Resin | 124 | 15 | 0.0164 | 1.24 | 1.91 | 1.54 |
| Cannabis Resin | 129 | 15 | 0.021 | 1.25 | 1.90 | 1.52 |
| Cannabis Resin | 134 | 15 | 0.0144 | 1.24 | 2.06 | 1.66 |

An input of cannabis resin was subjected to varying temperatures (210° C., 215° C. and 216° C.) and feed speeds (2.5 Hz, 5 Hz and10 Hz) with relatively consistent vacuum. Without being bound by any particular theory, enrichment of CBD: THC in the final distillate product increased from input material in comparison to the standard operating conditions. By increasing the temperature, the ratio of CBD:THC in the final distillate decreased slightly as compared to the resin input. Decreasing the feed speed also had a similar impact on enrichment, with the ratio of CBD:THC decreasing significantly from 5 Hz to 2.5 Hz. Without being bound by any particular theory, this increased ratio of CBD:THC in the distillate can be maintained at higher pressures by a corresponding increase in temperature. Table 9 summarizes the enrichment results.

TABLE 9
Impact of Temperature and Feed Speed on CBD:THC
Enrichment Under Increased Pressure Conditions

| | Temperature | Feed Speed | Vacuum | Input Ratio | Output Ratio | Enrichment Ratio |
|---|---|---|---|---|---|---|
| Material | (C.) | (HZ) | (mbar) | CBD:THC | CBD:THC | CBD:THC |
| Cannabis Resin | 210 | 10 | 13.3 | 1.24 | 1.98 | 1.60 |
| Cannabis Resin | 215 | 10 | 13.3 | 1.19 | 1.83 | 1.53 |
| Cannabis Resin | 216 | 10 | 13.3 | 1.19 | 1.83 | 1.53 |
| Cannabis Resin | 210 | 5 | 13.3 | 1.19 | 1.79 | 1.5 |
| Cannabis Resin | 210 | 5 | 10.6 | 1.23 | 1.84 | 1.49 |
| Cannabis Resin | 210 | 2.5 | 13.3 | 1.23 | 1.34 | 1.09 |

### Example 11

### High Boiling-Point Carrier Agent Impact on CBD:THC Enrichment

An input mixture of hemp resin was combined with canola oil or avocado oil and distilled at consistent operating conditions with lower than normal feed speed. Without being bound by any particular theory, enrichment of CBD:THC in the final distillate product decreased with a slight decrease in vacuum, from a ratio of 0.91 at 0.008 mbar, to 0.71 at 0.007 mbar. Table 10 summarizes the enrichment results.

TABLE 10
Impact of Oil Type on CBD:THC Enrichment

| Oil Type - Material | Temperature | Feed Speed | Vacuum | Input Ratio | Output Ratio | Enrichment Ratio |
|---|---|---|---|---|---|---|
| Type | (C.) | (Hz) | (mbar) | CBD:THC | CBD:THC | CBD:THC |
| Canola - Hemp Resin | 169 | 10 | 0.008 | 8.8 | 8.0 | 0.91 |
| Canola - Hemp Resin | 169 | 10 | 0.0075 | 7.8 | 6.6 | 0.85 |
| Canola - Hemp Resin | 169 | 10 | 0.007 | 8.4 | 6.0 | 0.71 |
| Avocado - Hemp Resin | 169 | N/A | N/A | 29.1 | 21.3 | 0.73 |

The data indicates that high boiling-point carrier agents (e.g. canola oil and avocado oil) may act to provide a reverse enrichment of CBD:THC in the distillate.

In the present disclosure, all terms referred to in singular form are meant to encompass plural forms of the same. Likewise, all terms referred to in plural form are meant to encompass singular forms of the same. Unless defined otherwise, all technical and scientific terms used herein have the same meaning as commonly understood by one of ordinary skill in the art to which this disclosure pertains.

As used herein, the term “about” refers to an approximately +/−10% variation from a given value. It is to be understood that such a variation is always included in any given value provided herein, whether or not it is specifically referred to.

It should be understood that the compositions and methods are described in terms of “comprising,” “containing,” or “including” various components or steps, the compositions and methods can also “consist essentially of” or “consist of” the various components and steps. Moreover, the indefinite articles “a” or “an,” as used in the claims, are defined herein to mean one or more than one of the element that it introduces.

For the sake of brevity, only certain ranges are explicitly disclosed herein. However, ranges from any lower limit may be combined with any upper limit to recite a range not explicitly recited, as well as, ranges from any lower limit may be combined with any other lower limit to recite a range not explicitly recited, in the same way, ranges from any upper limit may be combined with any other upper limit to recite a range not explicitly recited. Additionally, whenever a numerical range with a lower limit and an upper limit is disclosed, any number and any included range falling within the range are specifically disclosed. In particular, every range of values (of the form, “from about a to about b,” or, equivalently, “from approximately a to b,” or, equivalently, “from approximately a-b”) disclosed herein is to be understood to set forth every number and range encompassed within the broader range of values even if not explicitly recited. Thus, every point or individual value may serve as its own lower or upper limit combined with any other point or individual value or any other lower or upper limit, to recite a range not explicitly recited.

Therefore, the present disclosure is well adapted to attain the ends and advantages mentioned as well as those that are inherent therein. The particular embodiments disclosed above are illustrative only, as the present disclosure may be modified and practiced in different but equivalent manners apparent to those skilled in the art having the benefit of the teachings herein. Although individual embodiments are discussed, the disclosure covers all combinations of all those embodiments. Furthermore, no limitations are intended to the details of construction or design herein shown, other than as described in the claims below. Also, the terms in the claims have their plain, ordinary meaning unless otherwise explicitly and clearly defined by the patentee. It is therefore evident that the particular illustrative embodiments disclosed above may be altered or modified and all such variations are considered within the scope and spirit of the present disclosure. If there is any conflict in the usages of a word or term in this specification and one or more patent(s) or other documents that may be incorporated herein by reference, the definitions that are consistent with this specification should be adopted.

Many obvious variations of the embodiments set out herein will suggest themselves to those skilled in the art in light of the present disclosure. Such obvious variations are within the full intended scope of the appended claims.

1. An apparatus for isolating one or more cannabinoids from an input mixture, the apparatus comprising:
(a) a mixing vessel that is configured to receive a first input mixture and to combine the first input mixture with a high boiling-point carrier agent to provide a second input mixture, wherein one or more cannabinoids in the first input mixture are extracted into the high boiling-point carrier agent in the second input mixture;
(b) a volatizing unit that is configured to receive and volatilize the second input mixture into a cannabinoid-containing vapor stream and a residue; and
(c) a distillation unit for receiving the cannabinoid-containing vapor stream, wherein the distillation unit is configured to separate a first cannabinoid within the cannabinoid-containing vapor stream from at least a second cannabinoid.

2. The apparatus of claim 1, wherein the first input mixture is a cannabis concentrate.

3. (canceled)

4. (canceled)

5. The apparatus of claim 1, wherein the high boiling-point carrier agent is a vegetable oil, corn oil, canola oil, safflower oil, avocado oil, mineral oil, silicone oil, quinolone, alkyl derivatives of quinolone, ethylene glycol, glycol ethers, phthalates, triphenylmethane, triphenylmethane derivatives, or any combination thereof.

6.-10. (canceled)

11. The apparatus of claim 1, wherein the distillation unit comprises a distillation column that defines a plenum that is configured to separate the first cannabinoid from the second cannabinoid, and optionally one or more additional cannabinoids.

12.-17. (canceled)

18. An apparatus for isolating one or more cannabinoids from an input mixture, the apparatus comprising:
(a) a volatizing unit that is configured to receive and volatilize a first input mixture into a cannabinoid-containing vapor stream and a residue;
(b) a mixing vessel that is configured to receive and to combine the cannabinoid-containing vapor stream with a high boiling-point carrier agent to provide a second input mixture, wherein one or more cannabinoids are extracted into the high boiling-point carrier agent in the second input mixture; and
(c) a distillation unit for receiving the second input mixture, wherein the distillation unit is configured to separate a first cannabinoid within the second input mixture from at least a second cannabinoid.

19. The apparatus of claim 18, wherein the first input mixture is a cannabis concentrate.

20. (canceled)

21. The apparatus of claim 18, wherein the high boiling-point carrier agent is a vegetable oil, corn oil, canola oil, safflower oil, avocado oil, mineral oil, silicone oil, quinolone, alkyl derivatives of quinolone, ethylene glycol, glycol ethers, phthalates, triphenylmethane, triphenylmethane derivatives, or any combination thereof.

22.-26. (canceled)

27. The apparatus of claim 18, wherein the distillation unit defines a plenum that is configured to separate the first cannabinoid from the second cannabinoid, and optionally one or more additional cannabinoids.

28.-33. (canceled)

34. A method of isolating one or more cannabinoids from an input mixture, the method comprising steps of:
(a) combining a first input mixture with a high boiling-point carrier agent to provide a second input mixture, wherein one or more cannabinoids from the first input mixture are extracted into the high boiling-point carrier agent in the second input mixture;
(b) volatilizing the second input mixture to provide a cannabinoid-containing vapor stream and a residue;
(c) conducting the cannabinoid-containing vapor stream to a distillation unit that separates a first cannabinoid within the cannabinoid-containing vapor stream from at least a second cannabinoid; and
(d) collecting a product comprising the first cannabinoid.

35. The method according to claim 34, wherein the first input mixture is a cannabis concentrate.

36. (canceled)

37. The method according to claim 35, wherein a majority component of the second input mixture is the cannabis concentrate.

38. The method according to claim 34, wherein the high boiling-point carrier agent is a vegetable oil, corn oil, canola oil, safflower oil, avocado oil, mineral oil, silicone oil, quinolone, alkyl derivatives of quinolone, ethylene glycol, glycol ethers, phthalates, triphenylmethane, triphenylmethane derivatives, or any combination thereof.

39.-44. (canceled)

45. The method according to claim 34, wherein conducting the cannabinoid-containing vapor stream to the distillation unit further separates the first cannabinoid from at least one or more additional cannabinoids.

46. (canceled)

47. The method according to claim 34, wherein the first cannabinoid is THC, Δ8-THC, trans-Δ10-THC, cis-Δ10-THC, THCV, Δ8-THCV, Δ9-THCV, CBD, CBDV, CBC, CBCV, CBG, CBGV, CBN, CBNV, CBND, CBNDV, CBE, CBEV, CBL, CBLV, CBT, or cannabicitran.

48. (canceled)

49. (canceled)

50. (canceled)

51. A method of isolating one or more cannabinoids from an input mixture, the method comprising steps of:
(a) introducing a first input mixture into a volatizing unit for separating the first input mixture into a cannabinoid-containing vapor stream and a residue;
(b) combining the cannabinoid-containing vapor stream with a high boiling-point carrier agent to provide a second input mixture, wherein one or more cannabinoids from the cannabinoid-containing vapor stream are extracted into the high boiling-point carrier agent in the second input mixture;
(c) conducting the second input mixture to a distillation unit that separates a first cannabinoid within the second input mixture from at least a second cannabinoid; and
(d) collecting a product that comprises the first cannabinoid.

52. The method according to claim 51, wherein the first input mixture is a cannabis concentrate.

53. (canceled)

54. The method according to claim 51, wherein the high boiling-point carrier agent is a vegetable oil, corn oil, canola oil, safflower oil, avocado oil, mineral oil, silicone oil, quinolone, alkyl derivatives of quinolone, ethylene glycol, glycol ethers, phthalates, triphenylmethane, triphenylmethane derivatives, or any combination thereof.

55.-60. (canceled)

61. The method according to claim 51, wherein conducting the cannabinoid-containing vapor stream to the distillation unit further separates the first cannabinoid from at least one or more additional cannabinoids.

62. (canceled)

63. The method according to claim 51, wherein the first cannabinoid is THC, Δ8-THC, trans-Δ10-THC, cis-Δ10-THC, THCV, Δ8-THCV, Δ9-THCV, CBD, CBDV, CBC, CBCV, CBG, CBGV, CBN, CBNV, CBND, CBNDV, CBE, CBEV, CBL, CBLV, CBT, or cannabicitran.

64. (canceled)

65. (canceled)

66. The method according to claim 51, which provides for an enrichment of THC over CBD as compared to a ratio of THC:CBD in the first input mixture.
